(19) United States
(12) Patent Application Publication
(10) Pub. No.: US 2024/0424302 A1
(43) Pub. Date: Dec. 26, 2024

(54) NEUROMODULATION SYSTEM

(71) Applicant: Onward Medical N.V., Eindhoven (NL)

(72) Inventor: Yoann Dumény, Lausanne (CH)

(73) Assignee: Onward Medical N.V,, Eindhoven (NL)

(21) Appl. No.: 18/339,085

(22) Filed: Jun. 21, 2023

(51) Int. Cl. A61N 1/36; A61N 1/372

(52) U.S. Cl. CPC A61N 1/36146; A61N 1/37211

(57) ABSTRACT

Embodiments of the present disclosure may include a neuromodulation system. The neuromodulation system can include an implantable pulse generator configured to provide neuromodulation to a patient based on a sequence of neuromodulation programs. The implantable pulse generator can include alternately accessible memories. The alternately accessible memories can enable the implantable pulse generator to store a second neuromodulation program while providing neuromodulation according to a first neuromodulation program. The IPG can then transition from providing stimulation according to the first neuromodulation program to providing stimulation according to the second neuromodulation program without stopping stimulation. The neuromodulation system can include a parameter memory. Stimulation parameter updates can be written to the parameter memory. The value of the parameter memory can be used to determine the next stimulation pulse. The IPG can accordingly support pulse-to-pulse changes in stimulation parameters.

### TECHNICAL FIELD

The disclosed systems and methods concern updating stimulation settings for neuromodulation and/or neurostimulation systems. In particular, the disclosed systems and methods concern updating stimulation programs and/or parameters without interrupting the delivery of stimulation.

### BACKGROUND

Neuromodulation and/or neurostimulation systems can address physical or neurological injuries, diseases, or conditions of a patient by providing stimulation to the patient. Conventional neurostimulation systems can be designed to provide stimulation according to a previously programmed configuration, either continuously or in response to satisfaction of a condition. Such designs may require that stimulation be stopped prior to updating stimulation settings (electrode configurations, amplitude, frequency, etc.).

### SUMMARY

The disclosed systems and methods relate to a stimulation updating system. The stimulation updating system can include a pulse generator (e.g., an implanted or external pulse generator) configured with alternatively accessible program memories. The pulse generator can be configured to update a second program memory with a second stimulation program without interrupting the execution of a first stimulation program stored in a first program memory. Furthermore, in some embodiments, the pulse generator can be configured to support updating at least one stimulation parameter while performing stimulation.

Embodiments of the present disclosure include a neuromodulation system. The neuromodulation system can include an implantable pulse generator. The implantable pulse generator can be configured to provide neuromodulation to a patient based on a sequence of neuromodulation programs. The implantable pulse generator can include alternately accessible memories. A second memory of the alternately accessible memories can be configured for storing a second neuromodulation program while the implantable pulse generator provides neuromodulation according to a first neuromodulation program stored in a first memory of the memories.

Embodiments of the present disclosure include a method for providing neuromodulation according to a sequence of neuromodulation programs. The method can include an operation of receiving a second neuromodulation program in the sequence of neuromodulation programs. The second neuromodulation program can be received by the implantable pulse generator and during provision of neuromodulation according to a first neuromodulation program in the sequence of neuromodulation programs. The first neuromodulation program can be read from a first memory of the implantable pulse generator. The method can include an operation of storing the second neuromodulation program. The second neuromodulation program can be stored in a second memory of the implantable pulse generator. The method can include an operation of providing neuromodulation according to the second neuromodulation program stored in the second memory. The neuromodulation can be provided by the implantable pulse generator and upon completion of the provision of the neuromodulation according to the first neuromodulation program.

It is to be understood that both the foregoing general description and the following detailed description are exemplary and explanatory only and are not restrictive of the disclosed embodiments, as claimed.

### BRIEF DESCRIPTION OF THE FIGURES

The drawings are not necessarily to scale or exhaustive. Instead, emphasis is generally placed upon illustrating the principles of the embodiments described herein. The accompanying drawings, which are incorporated in and constitute a part of this specification, illustrate several embodiments consistent with the disclosure and, together with the description, serve to explain the principles of the disclosure. In the drawings:

FIG. 1 depicts an exemplary system for providing stimulation to one or more patients, according to some embodiments of the present disclosure.

FIG. 2 depicts an exemplary schematic of a pulse generator, according to some embodiments of the present disclosure.

FIG. 3 depicts an exemplary process for providing updated stimulation programs without ceasing stimulation, according to some embodiments of the present disclosure.

FIG. 4 depicts an exemplary process for updating stimulation parameters while a stimulation program is executing, according to some embodiments of the present disclosure.

FIG. 5 depicts an exemplary logical diagram of state machines, according to some embodiments of the present disclosure.

### DETAILED DESCRIPTION

Reference will now be made in detail to exemplary embodiments, discussed with regards to the accompanying drawings. In some instances, the same reference numbers will be used throughout the drawings and the following description to refer to the same or like parts. Unless otherwise defined, technical or scientific terms have the meaning commonly understood by one of ordinary skill in the art. The disclosed embodiments are described in sufficient detail to enable those skilled in the art to practice the disclosed embodiments. It is to be understood that other embodiments may be utilized and that changes may be made without departing from the scope of the disclosed embodiments. Thus, the materials, methods, and examples are illustrative only and are not intended to be necessarily limiting.

Conventional neurostimulation systems may require that stimulation be stopped prior to updating stimulation parameters. However, this requirement may pose a problem for patients. Patients that rely on stimulation for the proper operation of nerves, organs, or other structures may be harmed when stimulation stops. Furthermore, stopping stimulation interrupt patient activities (e.g., standing, locomotion, reaching and grasping, or the like) with potentially catastrophic consequences. Accordingly, updating stimulation settings may be a significant, potentially dangerous act for such patients. Patients may therefore choose to update stimulation settings infrequently, or only under clinician supervision, limiting the benefit provided by conventional neurostimulation systems.

Conventional neurostimulation systems may be unable to provide complicated patterns of stimulation. Implantable neurostimulators, in particular, can be limited to simple stimulation patterns by memory or processing constraints. As described herein, a neurostimulation system can address this problem using a programmer or controller in combination with an implantable neurostimulator. The programmer or controller can be programmed with one or more stimulator partitures. These stimulation partitures can include multiple channels of stimulation. The programmer or controller can select and provide the appropriate stimulation partiture to the implantable neurostimulator as needed. In this manner, the neurostimulation system can circumvent the memory or processing constraints of the implantable neurostimulator. However, this architecture may require that the implantable neurostimulator be able to update stimulation parameters while continuing to provide stimulation.

Emerging neurostimulation techniques can use complicated stimulation patterns to provide greater therapeutic benefits to patients. For example, such stimulation patterns can target individual leg muscles with different patterns of stimulation at different times in a gate cycle or stand-and-transfer motion. As an additional example, such stimulation patterns could enable bladder contractions while also mitigating autonomic dysreflexia triggered by a concomitant increase in bladder pressure. Conventional neurostimulation systems that cannot update stimulation parameters without ceasing stimulation may not support these beneficial emerging techniques.

The disclosed embodiments enable a neurostimulator to update stimulation parameters while continuing to provide stimulation. The disclosed embodiments can therefore benefit patients by preventing harms arising from cessation of stimulation and by enabling improved neurostimulation systems. The improved neurostimulation systems can support complicated stimulation patterns that provide greater therapeutic benefits.

Stimulation, as described herein, can include electrical stimulation. In some embodiments, stimulation can include optical, mechanical, or other suitable stimulation modalities.

Neuromodulation can include using stimulation to affect or control the operation or behavior of the nervous system, cardiac system, vascular system, gastrointestinal system, secretory system, musculature, or other excitable tissues. Neuromodulation can be used to restore function lost due to, mitigate the effects of, or compensate for injuries (e.g., spinal cord injury, stroke, or the like), diseases, disorders, or dysfunctions, including but not limited to Parkinson's Disease, Alzheimer's Disease, Huntington's Disease, Cerebral Palsy, Dystonia, Amyotrophic Lateral Sclerosis, Multiple Sclerosis. Particular applications of neuromodulation include, but are not limited to, spinal cord injuries, neurodegenerative disorders, pain, spasticity, movement disorders, epilepsy, ischaemia, cardiac disorders, bowel and bladder and sexual dysfunction, visual system disorders, auditory system disorders, and psychiatric disorders.

Consistent with disclosed embodiments, stimulation can be provided at various locations on or in the body of a patient. Stimulation can be provided to the central nervous system (e.g., within the brain and spinal cord), the peripheral nervous system, the secretory system, or the like. Stimulation can be configured to affect the sensory nervous system, the motor nervous system, the autonomic nervous system, the entric nervous system, or any combination of the forgoing. Stimulation can be provided to the spinal cord, spinal roots, dorsal root ganglions, or the like. Stimulation can be provided to particular tracts or lamina within the spinal cord. Stimulation can be provided to particular structures within the brain or brainstem, such as particular regions (e.g., thalamus, or the like), lobes (e.g., occipital lobe, or the like), or nuclei (e.g., globus pallidus or the like). Stimulation can be provided directly to muscles (e.g., using intramuscular electrodes, or the like) or sensory organs (e.g., the retina, the cochlea, carotid baroreceptors, or the like). The disclosed embodiments are not intended to be limited to any particular location or type of stimulation.

A patient, as described herein, can be the individual to whom stimulation is provided. A user, as described herein, can be the patient, a caregiver of the patient, or a service provider (e.g., a technician; a clinician, such as a doctor, nurse, rehabilitation specialist, physical therapist, or the like; or other service provider).

Stimulation parameters, as described herein, can include intensity, amplitudes, durations, frequency, carrier frequency, pulse width, pulse ration, electrode configuration, burst frequency or the like of the stimulation. In some embodiments, inter-burst interval; number of stimulation pulses in a burst; inter-pulse interval; first phase stimulation amplitude; first phase stimulation duration; first phase pulse shape, or the like. In some embodiments, stimulation parameters can include inter-phase duration and the shapes, amplitudes, durations, or the like of any subsequent phases in a stimulation pulse (e.g., second phase stimulation amplitude, second phase stimulation duration, second phase stimulation pulse shape, or the like). In some embodiments, stimulation parameters can include the choice of stimulation contacts (e.g., which electrode is used on a multi-contact electrode); stimulation polarity (e.g., monopolar, bipolar, or the like); whether the stimulation phases are charge-balanced, whether the stimulation is current, or voltage controlled, or the like.

A stimulation partiture can include an arrangement of stimulation parameters for multiple stimulation channels. The stimulation partiture can specify a temporal arrangement of the stimulation parameters. For example, stimulation according to a first set of stimulation parameters can be applied to a first stimulation channel according to a first schedule, while stimulation according to a second set of stimulation parameters can be applied to a second channel according to a second schedule.

A stimulation program can specify one or more sets of stimulation parameters, one or more stimulation partitures, or any combination of the foregoing. In some embodiments, the stimulation program can package the set(s) of stimulation programs or stimulation partiture(s) for provision to the patient as a single unit. In some embodiments, a stimulation program can be created, modified, or deleted using a programmer or controller, as described herein.

In some embodiments, a stimulation program can be designed and intended to provide a particular physiological effect or effects. For example, a stimulation program can include a stimulation partiture that specifies stimulation on channels corresponding to different muscles of the feet, legs, and trunk. The stimulation partiture can define sequences of stimulation times and/or stimulation amplitudes for these stimulation channels. In this example, when stimulation is provided according to the stimulation partiture by a stimulation engine through a suitably configured arrangement of leads to a paraplegic patient, the paraplegic patient may rise to a standing position from a seated position. As may be appreciated, such a positional change requires a complex combination of muscle activations.

In some embodiments, a sequence of stimulation programs can be designed and intended to collectively provide a particular physiological effect or effects. For example, a sequence of stimulation programs can be designed such that, when the stimulation programs are sequentially provided to and executed by a by a stimulation engine through a suitably configured arrangement of leads to a paraplegic patient, the paraplegic patient can rise from a sitting position (e.g., according to a first stimulation program in the sequence) and walk (e.g., according to one or more second stimulation programs in the sequence).

The physiological effect or effects can include locomotor activity, postural control, motor activities, voluntary voiding of the bladder and/or bowel, sexual function, and autonomic nervous system control. Locomotion activity can include activities that enable the patient to move from one location to another (e.g., walking, running, crawling, or the like). Motor activities can include voluntary motions, such as movements of the upper or lower limbs, neck, shoulders, hands, fingers (e.g., grasp, pinch, hold, or the like), feet, or the like. Postural control can include maintaining, achieving, or restoring a pose, posture, or balance at rest or in motion. Autonomic function control can include maintenance or control of the sympathetic, parasympathetic, or entric nervous system. For example, autonomic function control can include maintenance or control of pupil dilation, cardiovascular function (e.g., heart rate, blood pressure, or the like), respiratory and/or coughing function, or normalized metabolic processes.

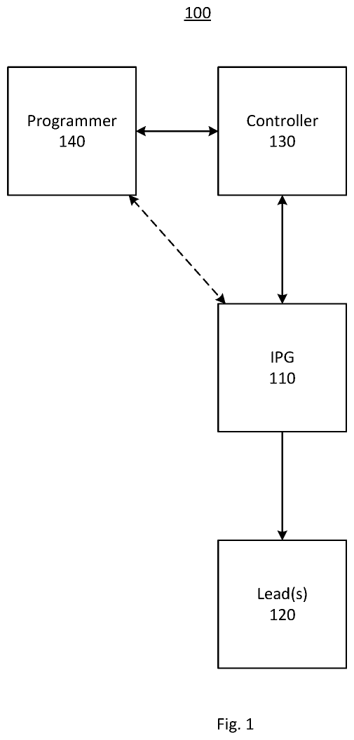

FIG. 1 depicts an exemplary system 100 for providing stimulation to one or more patients, according to some embodiments of the present disclosure. System 100 can enable flexible, customizable provision of stimulation to the patient. For convenience of description, system 100 is described as providing electrical stimulation, but the disclosed embodiments are not so limited. Furthermore, the stimulation provided can be used for neuromodulation. System 100 can therefore provide neuromodulation stimulation.

In some embodiments, neuromodulation system 100 can include an implantable pulse generator (IPG) 110, leads 120, a controller 130, and a programmer 140. IPG 110 can be configured to generate the electrical signals that are provided to the patients through leads 120. Leads 120 can be the physical interface through which the stimulations are provided to the patient. Controller 130 can be a patient-facing computing device and can enable a patient or a user to enable, disable, trigger, halt, or adjust stimulation provided by IPG 110 in accordance with a predetermined stimulation program. Programmer 140 can be a user-facing computing device and can be configured to enable a user to create, modify, or delete stimulation programs (e.g., by creating, modifying, or deleting sets of stimulation programs or partitures specified by a stimulation program).

In various embodiments, programmer 140 and external controller 130 can be implemented using the same computing device. For example, such a device can have multiple accounts having different privileges, such as a patient account and a user account. In such embodiments, a patient account can provide the functionality of external controller 130, while a user account can provide the functionality of programmer 40.

In various embodiments, an implantable controller can be used in place of external controller 130. In some such embodiments, the implantable controller 130 and IPG 110 can be combined in a single device. Alternatively, the implantable controller 130 and IPG 110 can be in separate devices.

In some embodiments, IPG 110 can be communicatively connected with leads 120. For example, leads 120 can be electrically connected to IPG 110 using connectors, as described herein. In some embodiments, IPG 110 can be communicatively connected with controller 130. In some embodiments, IPG 110 can be communicatively connected with programmer 140. IPG 110 can be configured to receive stimulation programs or instructions from controller 130 or programmer 140 and provide stimulation pulses to the patients through leads 120 according to a stimulation program.

In various embodiments, an external pulse generator can be used in place of IPG 110. For example, an external pulse generator connected to percutaneous leads can be used to determine whether a patient is responsive to stimulation. If the patient is responsive, then the patient can be outfitted with an implantable stimulator. In some such embodiments, the external pulse generator can possess functionality of implantable pulse generator 110, as described herein. Alternatively, the external pulse generator can additionally include the capabilities of controller 130 (or programmer 140).

In some embodiments, the external pulse generator and one or more of the external controller and programmer can be implemented using a single device. For example, a single device can provide the functionality of the pulse generator and the external controller, or the pulse generator, external controller, and programmer. Alternatively, the external pulse generator, external controller, and programmer can be implemented using separate devices.

Consistent with disclosed embodiments, IPG 110 can be configured to provide stimulation signals on stimulation channels. In some embodiments, the stimulation channels can be independently controllable. IPG 110 can be configured to receive instructions or a stimulation program from at least one of controller 130 or programmer 140. Such instructions can include instructions to load a stimulation program (or to load parameters that specify a stimulation program) into a memory of IPG 110. The instructions can include instructions to modify a stimulation program or delete a stimulation program from a memory of IPG 110. The instructions can include instructions to enable or disable an output of IPG 110. The instructions can include instructions to start or cease stimulation according to a stimulation partiture.

For example, IPG 110 can receive (e.g., from controller 130 or programmer 140) a first stimulation program (or parameters specifying the stimulation program) corresponding to a component of a gait cycle. IPG 110 can provide stimulation to the patient according to the stimulation program. While providing stimulation according to the first stimulation program, IPG 110 can receive another stimulation program corresponding to the next component of the gait cycle. Upon completion of stimulation according to the first stimulation program, IPG 110 can provide stimulation according to the second stimulation program. As a result of the sequential execution of such stimulation programs, the patient can move, be moved, or be assisted in moving, through a gait cycle.

In some embodiments, IPG 110 can be configured for implantation into a patient. IPG 110 can include control circuitry, communication circuitry, a stimulation engine, and a connection component. The control circuitry can include at least one processor (e.g., a microprocessor, a microcontroller, an Application Specific Integrated Circuit, or another suitable processor) and at least one memory containing instructions that control the operation of IPG 110. In some embodiments, the communication circuitry can be configured to support wireless communication with controller 130 or programmer 140. In such embodiments, the communication circuitry can include an antenna (e.g., a radio-frequency antenna, such as an antenna coil, or the like), an optical link (e.g., an optical detector, or the like), or any other suitable communication components. In some embodiments, the communication circuitry can support wired communication with controller 130 or programmer 140. In such embodiments, communication circuitry can include suitable connectors and/or connectors for created wired percutaneous connections with other components of system 100. The connection component can enable IPG 110 to connect with leads (e.g., leads 120) to provide stimulation through the leads without unacceptable signal distortion or power loss. The disclosed embodiments are not limited to any particular interconnection architecture.

In some embodiments, IPG 110 can include a power source. In some embodiments, the power source can be configured to store power for operating IPG 110 within the device (e.g., a battery, a fuel cell, or another suitable power source). In some embodiments, the power source can be configured to obtain power from an external source (which can then be stored internally, in some embodiments). For example, IPG 110 can include a radiofrequency coil for receiving externally transmitted power.

In some embodiments, IPG 110 can include a stimulation engine. In some embodiments, the stimulation engine can be configured to convert control signals into stimulation signals that can be provided to the patient. In some embodiments, the control signals can be logic signals, an array or time series of digital data, or any other suitable control signal. For example, when the stimulation is electrical stimulation, the stimulation engine can be or include one or more amplifier(s). The input to the amplifier(s) can be nanowatt or microwatt control signals and the output of the amplifier can be milliwatt voltage-controlled or current controlled-stimulation signals. The stimulation engine can include outputs corresponding to the multiple stimulation channels of IPG 110.

In some embodiments, the components of IPG 110 can be contained inside a container. The container can be sealed (e.g., hermetically sealed) to prevent or reduce communication between the environment (e.g., the implantation site within the patient) and the interior of IPG 110. In some embodiments, the container can include an electrically conductive portion. The electrically conductive portion can serve, in some instances, as a return path for current, or as a ground reference for electrical stimulation.

In some embodiments, the neuromodulation system can provide stimulation to a patient using leads 120. Leads 120 can be the interface between implantable pulse generator 110 and the patient's tissue. Leads 120 can include connectors, wiring or cabling, and stimulation contacts. Connectors can be configured to interconnect with corresponding connectors on IPG 110. Wiring or cabling can convey stimulation signals from IPG 110 to the stimulation contacts. The particular implementation of the connectors, wiring or cabling, and stimulation contacts can depend on the stimulation modality. For example, when the stimulation is electrical stimulation, the stimulation contacts can be or include electrodes (which can have contact(s)). The wiring or cabling can provide electrical connectivity from IPG 110 to the electrodes. The leads 120 can be electrically connected or communicated with implantable pulse generator 110.

The disclosed embodiments are not limited to any particular electrode design. In some embodiments, leads 120 may have optimal electrode configuration on one end and implantable pulse generator 110 on the other end. In particular, leads 120 is not limited to the size, shape or number. The electrode can be configured to place directly on nerves, or the electrode can be configured to place next to the nerves. Suitable electrode designs can include array or microarray electrodes, paddle electrodes; lead electrodes including circumferential contact(s), side contact(s), or tip contact(s); cuff electrodes; wire or microwire electrodes; or any other suitable electrode design.

The disclosed embodiments are not limited to any electrodes designed for any particular purpose. Suitable electrodes include brain-computer interface electrodes, deep brain stimulation electrodes, cochlear stimulation electrodes, retinal stimulation electrodes, vagal stimulation electrodes, spinal cord stimulation electrodes, foramen stimulation electrodes, bladder or bowel control stimulation electrodes, intramuscular stimulation electrodes, efferent stimulation electrodes, afferent stimulation electrodes, autonomic stimulation electrodes, enteric stimulation electrodes, or electrodes designed for some other suitable purpose.

Consistent with disclosed embodiments, controller 130 can be configured to enable a patient or user to enable, disable, trigger, halt, or adjust stimulation provided by IPG 110. In some embodiments, controller 130 can be an external device, while IPG 110 is implanted in the patient. In some embodiments, controller 130 can have greater processing power, memory, power usage, or the like than IPG 110. In some embodiments, controller 130 can include input/output options that IPG 110 lacks, such as physical communication ports (e.g., USB, RS232, or ethernet ports, or the like) or a display.

Consistent with disclosed embodiments, controller 130 can extend the capabilities of IPG 110. In some embodiments, controller 130 can store one or more stimulation programs for use by IPG 110. In some embodiments, controller 130 can be configured with data or instructions governing the selection and provision of stimulation programs to IPG 110. For example, controller 130 can store execution conditions or relationships for stimulation programs. Controller 130 can provide stimulation programs to IPG 110 in response to, or in accordance with, these execution conditions or relationships. In this manner, controller 130 can circumvent memory or computational limitations of IPG 110.

Consistent with disclosed embodiments, controller 130 can be configured to provide instructions to IPG 110. These instructions can include instructions to IPG 110 to load a stimulation program, modify or delete a stimulation program, enable or disable a stimulation program of IPG 110, start or cease execution of a loaded stimulation program, or at any other appropriate trigger. For example, controller 130 can receive instructions from programmer 140 that define a set of stimulation programs corresponding to a gait cycle. The instructions can also specify an execution relationship: that these stimulation programs can be provided to IPG 110 in an appropriate sequence. Then, in response to some trigger (e.g., a patient interaction with an interface of controller 130), controller 130 can provide the stimulation programs to IPG 110 in the sequence. Controller 130 can provide the stimulation partitures such that the next stimulation partiture in the gait cycle is being loaded into IPG 110, while IPG 110 is providing stimulation according to the current stimulation program in the gait cycle.

Consistent with disclosed embodiments, controller 130 can receive instructions from programmer 140. The instructions can include instructions to load a stimulation program into a memory of controller 130. The instructions can include instructions to modify or delete a stimulation program from a memory of controller 130. In some embodiments, the instructions received from programmer 140 can define sets of stimulation parameters, programs and execution conditions or relationships among these stimulation programs.

As an additional example, controller 130 can receive instructions from programmer 140 that specify a stimulation program for mitigation of autonomic dysreflexia. The instructions can also specify an execution relationship: that this stimulation program be provided to IPG 110 if the blood pressure of the patient exceeds a certain threshold level.

In some embodiments, controller 130 can include a user interface. A patient can interact with the user interface to cause controller 130 to perform certain actions. In some embodiments, these actions can depend on the instructions received by controller 130 from programmer 140. For example, the patient can cause controller 130 to provide stimulation programs received from programmer 140 to IPG 110. In some embodiments, these actions can depend on the state of IPG 110. For example, the patient can cause controller 130 to instruct IPG 110 to execute (or cease execution of) a stimulation partiture loaded onto IPG 110. The disclosed embodiments are not limited to any particular user interface implementation. In some embodiments, the user interface can be or include a graphical user interface (e.g., a screen, touchscreen, or the like), an audio user interface, a mechanical user interface (e.g., pushbutton(s), switch(es) or the like), or other suitable user interfaces.

In some embodiments, controller 130 can be configured to maintain a communication link with IPG 110. In some embodiments, the communication link can be a wireless link. For example, the communication link can be a WiFi link, a Bluetooth link, an optical link, a wireless radiofrequency telemetry link, or any other suitable link. In some embodiments, the communication link can be a wired link. The communication link can be used to exchange data and/or instructions between controller 130 and IPG 110.

As may be appreciated, the communication link may fail due to interference, a change in the relative positions of controller 130 and IPG 110, the interposition of material between controller 130 and IPG 110, a hardware or software issue in either of controller 130 or IPG 110. The disclosed embodiments are not limited to any particular reason for such a failure.

In various embodiments, controller 130 or IPG 110 can be configured to detect a failure of the communication link between controller 130 and IPG 110. Controller 130 or IPG 110 can detect a failure of the communication link using a keepalive mechanism. For example, controller 130 can provide a keepalive message to IPG 110. IPG 110 can respond to receipt of a keepalive message with a confirmation message. Receipt of the confirmation message by controller 130 can indicate that the communication link remains active (and that IPG 110 is still working). Controller 130 can repeatedly provide keepalive messages to IPG 110 (e.g., periodically or according to a schedule). As may be appreciated, in some embodiments, IPG 110 can provide the keepalive message and controller 130 can provide the confirmation message.

In some embodiments, IPG 110 can be configured to determine that the communication link has failed when it does not receive a keepalive (or confirmation) message for a predetermined period of time. In some embodiments, the period of time can be between 0.1 seconds and 5 minutes. In various embodiments, the period of time can be between 0.1 seconds and 60 seconds. In some embodiments, IPG 110 can be configured to cease providing stimulation in response to a determination that the communication link has failed. For example, when controller 130 is configured to provide IPG 110 a keepalive message every second, IPG 110 has not received a keepalive message for three seconds, and the period of time is 3 seconds, then IPG 110 can stop providing stimulation.

In some embodiments, controller 130 can be configured to determine that the communication link has failed when it does not receive a keepalive (or confirmation) message for a predetermined period of time. In such embodiments, controller 130 can notify programmer device 140 that the communication link has failed (or that the stimulation has been stopped). In some embodiments, controller 130 can notify programmer device 140 that the communication link previously failed, should IPG 110 subsequently reestablish the communication link. For example, controller 130 can track the occurrence and duration of communication link failures and subsequently provide this information to programmer 140.

Consistent with disclosed embodiments, programmer 140 can be configured to enable a user to create or arrange stimulation programs for a patient. In some embodiments, creation of a stimulation program can include the selection or adjustment of spatial and temporal parameters for stimulation. Such selection or adjustment can include the selection or adjustment of stimulation channels used, the mapping stimulation channels to particular electrodes in the patient, or the timing of stimulation on different stimulation channels. Such selection or adjustment can further include the selection or adjustment of stimulation parameters. In some embodiments, creation of a stimulation partiture can involve using another stimulation partiture as a template or basis. The other stimulation program can be a default stimulation program, a stimulation program used by another patient, or a stimulation program previously used by the current patient. Creation of the stimulation program for the current patient can include obtaining and adjusting the other stimulation program. The other stimulation program can be obtained from another device (e.g., controller 130, IPG 110, a database, another programmer, or the like).

In some embodiments, arrangement of stimulation programs can include the creation of execution relationships among stimulation programs. In some instances, a user can interact with programmer 140 to create a sequence of stimulation programs that are executed sequentially by IPG 110. In some instances, a user can interact with programmer 140 to associate conditions with the execution of the stimulation program. In various embodiments, programmer 140 can associate stimulation programs with user interface elements of controller 130 (e.g., pushbuttons or toggles), such that a patient interaction with the user interface elements can cause execution of the stimulation program.

As may be appreciated, programmer 140 can provide instructions configuring controller 130. Such instructions can include stimulation partitures and execution relationships. Furthermore, in some embodiments, programmer 140 can provide instructions directly to IPG 110. These instructions can include instructions to IPG 110 to load a stimulation program, modify or delete a stimulation program, enable or disable a stimulation partiture of IPG 110, start or cease execution of a loaded stimulation program, or the like. In this manner, a user can interact with programmer 140 to determine appropriate stimulation programs for a patient. Once the stimulation programs are determined, the user can transmit the determined stimulation programs (and any execution relationships) to controller 130. The patient (or a user) can then interact with controller 130 to cause IPG 110 to provide the intended stimulation.

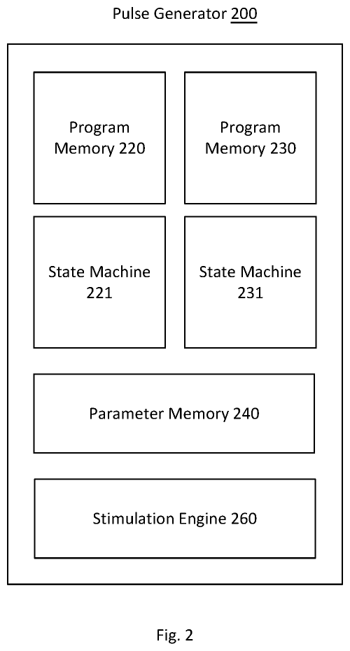

FIG. 2 depicts an exemplary schematic of a pulse generator 200, consistent with disclosed embodiments. Pulse generator 200 can be an external or implantable pulse generator, as described with regards to FIG. 1. In some embodiments, pulse generator 200 can be configured to include program memory 220 and program memory 230, state machine 221 and state machine 231, parameter memory 240 and stimulation engine 260. As may be appreciated, pulse generator 200 is not limited to two program memories and two state machines. Consistent with disclosed embodiments, program memory 220 and program memory 230 and state machine 221 and state machine 231 can enable pulse generator 200 to load a second stimulation program while executing the stimulation according to a first stimulation program. As may be appreciated, stimulation provided by pulse generator 200 can be used for neuromodulation.

In some embodiments, program memory 220, program memory 230 can be implemented using separate components (e.g., separate memory modules, or sets of memory modules). In some embodiments, program memory 220, program memory 230 can be implemented using separate logical areas in a unified memory (e.g., a single memory module). In such embodiments, the unified memory can be configured to support simultaneous reading and writing operations. Consistent with disclosed embodiments, each of program memory 220 and program memory 230 can be configured to store at least one stimulation program.

In some embodiments, program memory 220 and program memory 230 can be alternately accessible. In some embodiments, for example, when program memory 220 is available for writing, program memory 230 can be unavailable for writing. One or more stimulation programs can then be written to program memory 220. In some embodiments, later written stimulation programs can overwrite previously written stimulation program. When program memory 230 becomes available for writing, program memory 220 can become unavailable for writing. One or more stimulation programs can then be written to program memory 230. In some embodiments, later written stimulation programs can overwrite previously written stimulation program. In some embodiments, a program memory can be available for reading when the program memory is unavailable for writing. Consistent with disclosed embodiments, pulse generator 200 can read from one memory while writing to the other memory.

In some embodiments, state machines 221 and 231 can control reading and writing to memories 220 and 230, respectively. State machines 221 and 231 can include four or more states as described herein. The states can include a first state (e.g., “ready to update”), a second state (e.g., “updating”), a third state (“ready to execute”), and a fourth state (“executing”). The state machines can implement specific, allowable transitions between these states, as described herein. Consistent with disclosed embodiments, the state machines can be implemented using any suitable architecture. The disclosed embodiments are not limited to any particular programming language or hardware implementation.

Consistent with disclosed embodiments, state machine 221 can control memory 220 and state machine 231 can control memory 230. In some embodiments, when state machine 221 is in the fourth state (e.g., a first stimulation program stored in program memory 220 is being accessed), state machine 231 can be in the first state (e.g., program memory 230 is configured to receive and store a stimulation program). When a second stimulation program is received (e.g., as part of an update request), state machine 231 can transition to the second state (e.g., storing the second stimulation program). Once the second stimulation program is stored, state machine 231 can transition to the third state. When state machine 231 is in the third state, receipt of an execution instruction can cause state machine 231 to transition to the fourth state. In the fourth state, the second stimulation program can be queued up for outputting. Once the reading and outputting of the first stimulation program is finished, the second stimulation program can be output from program memory 230 without interrupting stimulation.

In some embodiments, when one state machine transitions from the second state to the third state, and the other state machine is already in the third state, the other state machine can transition to the first state. For example, state machine 231 can be in the third state (e.g., “ready to execute”) and state machine 221 can be in the first state (e.g., “ready to update”). State machine 221 can receive an update request and transition to the second state (e.g., “updating”). When state machine 221 completes updating and transitions to state three, state machine 231 can transition to state one, unless state machine 231 has already transitioned to state four. If state machine 231 has already transitioned to state four, then state machine 231 can transition to state one once the second stimulation program has ceased executing.

In some embodiments, when one state machine is in the fourth state and the other state machine is not in the third state, completion of stimulation can cause the state machine in the fourth state to return to the third state. Receipt of additional execution requests can cause the state machine to return to the fourth state, retriggering stimulation. For example, state machine 231 can be in the fourth state and state machine 221 can be in the first state. Upon cessation of stimulation, state machine 231 can return to the third state. Should pulse generator 200 receive another execution request prior to cessation of stimulation, state machine 231 can return to the fourth state, retriggering stimulation.

In some embodiments, priorities can be associated with transitions between states. For example, the transitions involving updating (e.g., from state one to state two, or from state two to state three) can be lower priority that the transitions involving execution (e.g., from state three to state four, from state four to state four, or from state four to state one). In some embodiments, transitions involving updates can be implemented as application layer functions, while transitions involving execution can be implemented as high priority tasks.

In some embodiments, pulse generator 200 can be configured to include a parameter memory 240. Parameter memory 240 can enable pulse-to-pulse modification of the stimulation characteristics of a currently executing stimulation program. Parameter memory 240 can be configured to store neuromodulation parameters. When a neuromodulation pulse is generated, the characteristics of the neuromodulation pulses can depend on values stored in parameter memory 240. Parameter memory 240 can be accessible during provision of stimulation such that the stimulation parameter can be changed from pulse to pulse. For example, stimulation amplitude can be stored in parameter memory 240. When a pulse is output, the amplitude of the pulse can depend on the parameter stored in parameter memory 240. In response to data or instructions received from another device (e.g., controller 130 or programmer 140), amplitude parameters in parameter memory 240 can be modified or overwritten. In this manner, pulse to pulse changes in stimulation amplitude can be achieved. As may be appreciated, other stimulation parameters can be similarly updated pulse to pulse.

Consistent with disclosed embodiments, stimulation engine 260 can be configured to control storing and execution of stimulation programs. In some embodiments, stimulation engine 260 can be configured to assign stimulation programs received (e.g., from programmer 140 and/or controller 130) to program memory. In some embodiments, stimulation engine 260 can be configured to update the states of the state machines. In some embodiments, stimulation engine 260 can maintain a queue of simulation programs (or memories). Stimulation engine 260 can read from the memory associated with the stimulation program at the head of the queue, thereby jumping from one stimulation program to the another seamlessly, without ceasing stimulation. As described herein, the stimulation output can be provided to leads 120.

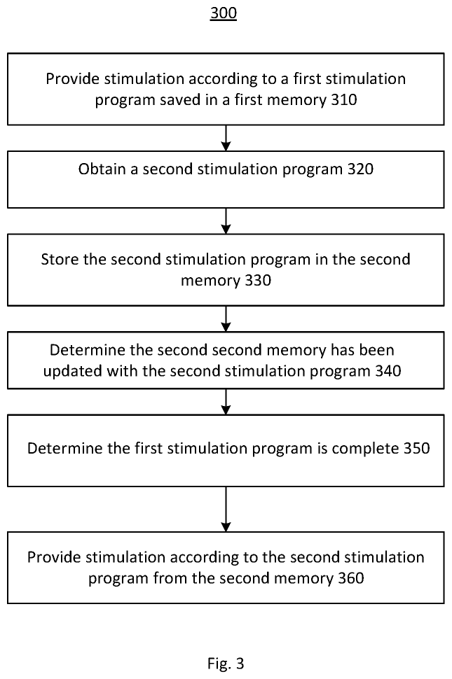

FIG. 3 depicts an exemplary process 300 for providing updated stimulation programs without ceasing stimulation, consistent with disclosed embodiments. As may be appreciated, stimulation provided according to the stimulation programs can be used for neuromodulation. For convenience of description, process 300 is described with reference to a pulse generator (e.g., IPG 110, pulse generator 200, or the like). The pulse generator can alternatively accessible memories. In some embodiments, the pulse generator can include two program memories (e.g., program memories 220 and 230), two state machines (e.g., state machines 221 and 231), and a stimulation engine (e.g., stimulation engine 260). However, this description is not intended to be limiting. In some embodiments, for example, process 300 can be performed using an external stimulator. Furthermore, the pulse generator can include additional memories, additional state machines, and/or additional stimulation engines.

Consistent with disclosed embodiments, process 300 can enable the pulse generator to receive stimulation programs (e.g., from an controller, such as controller 130, or a programmer, such as programmer 140) without ceasing stimulation. The received stimulation program can be stored in the currently accessible program memory. In response to an instruction to execute the newly stored stimulation program, the stimulation program can be queued up by the stimulation engine. Upon completion of stimulation according to the currently executing stimulation program, the stimulation engine can begin providing stimulation according to the newly stored stimulation program, without ceasing stimulation.

In step 310 of process 300, the pulse generator can provide stimulation to a patient, consistent with disclosed embodiments. The stimulation can be provided in accordance with a first stimulation program. The first stimulation program can be saved in a first program memory of the pulse generator.

In step 320 of process 300, the pulse generator can obtain a second stimulation program, consistent with disclosed embodiments. In some embodiments, obtaining the second stimulation program can include receiving an instruction to obtain the second stimulation program. The instruction can be received by the pulse generator from a device connected by a communication link to the pulse generator (e.g., controller 130, programmer 140, or the like). The instruction can indicate that the pulse generator should obtain a second stimulation program. In some embodiments, the instruction can further indicate that the pulse generator should provide stimulation according to the second stimulation program.

In some instances, the second stimulation program may have been created or modified using a programmer (e.g., programmer 140, or the like) or another device (e.g., controller, computer, smart phone, tablet, or the like). The second program may have been created or modified using a graphical user interface. In some embodiments, the second stimulation program can enable a different action, or a different series of actions, compared with the first stimulation program. In some embodiments, the second stimulation program can be set up with different parameters to do the same or similar actions as the first stimulation program.

In some embodiments, the instruction can be provided by the device in response to a user or patient input (e.g., an interaction with a graphical user interface to begin a stimulation program) or in accordance with execution conditions or relationships. For example, an execution condition can specify provision of the second stimulation program in response to a certain status of the patient (e.g., provision of a stimulation program configured to reduce blood pressure in response to detection of high patient blood pressure). As an additional example, an execution relationship can specify that the first stimulation program is one of a sequence of such programs and the second stimulation program is the next stimulation program in that sequence. The sequence of stimulation programs (which may include loops, conditionals, or other flow control instructions) can enable the patient to perform complicated actions (e.g., standing on a chair) or a series of repetitive actions (e.g., walking by enabling alternating leg movement).

In some embodiments, in response to the instruction, the pulse generator can retrieve the second stimulation program from the device (or another source). For example, the pulse generator can provide a response to the device requesting transmission of the second stimulation program. In various embodiments, the second stimulation program can be provided together with the command, or following the command. In such embodiments, the pulse generator may not provide a response to the command. In some embodiments, the second stimulation program can be or include the command. For example, provision of the second stimulation program can constitute a command to execute the second stimulation program.

Consistent with disclosed embodiments, the stimulation engine can store the obtained second stimulation program in the currently accessible second program memory. The pulse generator can continue to provide stimulation according to the first stimulation program while the second program memory is being updated.

In step 330 of process 300, the pulse generator can store the second neuromodulation program in the second program memory, consistent with disclosed embodiments. As described with regards to FIG. 2, the pulse generator can update the state machine for the second program memory, transitioning the state machine from the first state (“ready to update”) to the second state (“updating”).

In step 340 of process 300, the pulse generator can determine that the second program memory has been updated with the second stimulation program, consistent with disclosed embodiments. As may be appreciated, the update to the second program memory may not be completed until the second stimulation program has been fully received by the pulse generator. In some instances, depending on the data rate of the communication link between the device providing the stimulation program and the pulse generator, the second stimulation program may be received within a relatively short period of time. In other instances, additional time may be required for the second stimulation program to be received. Any suitable method can be used to determine that the update to the second program memory has been completed. In response to the completion of the update to the second program memory, the pulse generator can update the state machine for the second program memory, transitioning the second state machine from the second state (“updating”) to the third state (“ready to execute”).

In step 350 of process 300, the pulse generator can determine that stimulation according to the first stimulation program has completed, consistent with disclosed embodiments. In some embodiments, a number of repeats (e.g., a repeat value) can be specified for the first stimulation program. For example, an instruction specifying the number of repeats may have been received together with the first stimulation program. In some embodiments, repeats can be implemented using a queue that schedules stimulation. The queue can include multiple sequential references to the first stimulation program. As repeats are completed, they can be removed from the queue and stimulation providing according to the next referenced stimulation program. In some embodiments, the pulse generator can use counters to track the number of repeats remaining. As repeats are completed, the associated counter can be decremented. In such embodiments, stimulation according to the first stimulation program can be completed when no repeats remain, and the stimulation specified by the final repeat of the first stimulation program is completed. When no repeat is scheduled, stimulation can be complete when the stimulation specified by the first stimulation program is completed. For example, the first stimulation program can specify current-controlled 20 Hz stimulation for 30 seconds in alternating 1 second on, 1 second off bursts on two stimulation channels. The pulse generator can be configured to provide three repeats of this stimulation program. Stimulation according to the first stimulation program can therefore be complete when such stimulation has been provided for 90 seconds. Alternatively, when no repeats are specified, stimulation according to the first stimulation program can be complete when such stimulation has been provided for 30 seconds. Once the stimulation according to the first stimulation program has completed, the pulse generator can transition the first state machine from the fourth state (“executing”) to the first state (“ready to update”).

In some embodiments, the programmer or controller can be configured to track the number of repeats of the first stimulation program. For example, the controller or programmer can be configured with a counter for tracking a number of repeats of the first stimulation program. In some embodiments, the counter can be initialized to a repeat value. After each repeat, the counter can be decremented. In such embodiments, when a value of the counter is not zero, the programmer or controller can provide an execution request to the pulse generator. In response to the execution request, the pulse generator can provide stimulation according to the first stimulation program. Alternatively, the counter can be initialized to zero. After each repeat, the counter can be incremented. In such embodiments, when the value of the counter is less than the repeat value, the programmer or controller can provide an execution request to the pulse generator. In this manner, the programmer or controller can ensure that the stimulation is provided for the specified number of repeats.

In step 360 of process 300, the pulse generator can provide stimulation according to the second stimulation program, consistent with disclosed embodiments. In some embodiments, the pulse generator can access the second program memory to read the second stimulation program and provide an output as specified by the second memory. Upon the start of stimulation, the pulse generator can transition the second state machine from the third state (“ready-to-execute”) to the fourth state (“executing”).

In some embodiments, the pulse generator can maintain a queue for scheduling stimulation. The queue can include references to program memories. The pulse generator can access the referenced memory to obtain a stimulation program. The pulse generator can the provide stimulation according to the stimulation program. In some embodiments, the pulse generator can access stimulation programs in the order in which they are received by the pulse generator.

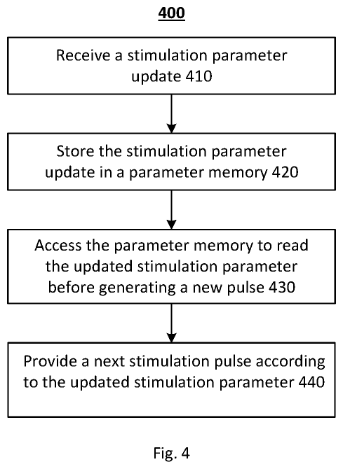

FIG. 4 depicts an exemplary process 400 for updating stimulation parameters while a stimulation program is executing, consistent with disclosed embodiments. Process 400 can enable pulse-to-pulse changes in stimulation parameters without stopping stimulation. For convenience of description, the process 400 is described with reference to a pulse generator (e.g., IPG 110, pulse generator 200, or the like) that implements a parameter memory (e.g., parameter memory 240, or the like) and a stimulation engine (e.g., stimulation engine 260, or the like). As may be appreciated, this description is not intended to be limiting. Process 400 can be performed using other architectures, or other stimulation devices. The stimulation provided can be used for neuromodulation, consistent with disclosed embodiments.

In step 410 of process 400, the pulse generator can receive an instruction to update at least one stimulation parameter. The instruction can be received by the pulse generator from a device connected by a communication link to the pulse generator (e.g., controller 130, programmer 140, or the like). In some embodiments, the instruction can be provided by the device in response to a user or patient input (e.g., an interaction with a graphical user interface to increase a stimulation amplitude or change a stimulation frequency). In some embodiments, the instruction can be provided by the device after an initial configuration of the currently executing stimulation program.

In some embodiments, the instruction can specify a value for the stimulation parameter (e.g., a stimulation amplitude value). In some embodiments, the instruction can specify an intermediate value then can be used to generate one or more values of one or more stimulation parameters. For example, the instruction can specify an incremental change in the value of a stimulation parameter (e.g., increase pulse width 0.010 ms, or the like). As an additional example, the instruction can specify a value or incremental change for a stimulation parameter that is related (e.g., by rules or conditions) to multiple other stimulation parameters, such that changing the one stimulation parameter changes the other stimulation parameters. For example, increasing a current amplitude may increase a charge per phase. The pulse generator may be programmed with limits on charge per phase, such that an instruction increasing the current amplitude results in a concomitant decrease in the pulse duration, such that the charge per phase value remains within the limit. As an additional example, when current controlled stimulation is provided to multiple electrodes, an overall stimulus amplitude can be converted to a per-electrode stimulus amplitude.

In step 420 of process 400, the pulse generator can store the stimulation parameter in the parameter memory, consistent with disclosed embodiments. In some embodiments, storing the stimulation parameter can include storing a function of the value of the stimulation parameter. For example, the instruction can specify a first stimulation amplitude. The currently executing stimulation program can specify a second stimulation amplitude. In this example, storing the stimulation parameter can include storing a ratio of these stimulus amplitude values in the parameter memory, such that the product of the stored value and the second stimulation amplitude equals the first stimulation amplitude.

In step 430 of process 400, the pulse generator can access the parameter memory during the provision of stimulation according to a stimulation program. In some embodiments, the value of the stimulation parameter can be accessed prior to generating the next stimulation pulse. Consistent with disclosed embodiments, the pulse generator can determine the appropriate values for the next stimulation pulse based at least in part on the stimulation pulse value stored in the parameter memory. In some embodiments, the value stored in the parameter memory can replace the corresponding value stored in the program memory. For example, the stimulation program can specify a first pulse width and the value of the stimulation parameter can specify a second pulse width. The next stimulation pulse can then be generated using the second pulse width. In some embodiments, the value stored in the parameter memory can be combined with the corresponding value stored in the program memory to generate the value for the next stimulation pulse. For example, when the stored stimulation parameter is a ratio of a stimulation amplitude specified in the received instructions and the stimulation amplitude specified in the stimulation program, the next stimulation pulse can then be generated using the product of the stored stimulation parameter and the stimulation amplitude specified in the stimulation program.

In step 440 of process 400, the pulse generator can provide a next stimulation pulse based on the updated neuromodulation parameter. In this manner, the programmer or controller can provide an instruction to change the stimulation parameter and a corresponding change in the stimulation parameter can occur within a short period of time. In some embodiments, this period of time can be approximately equal to (or less than) the sum of the time required to communicate the instruction and the interpulse interval of the currently executing stimulation program. In some embodiments, the period of time required to change the stimulation can be less than 100 ms, less than 50 ms, less than 30 ms, or less.

Consistent with disclosed embodiments, a reset of all the updated parameters can be available via the user interface. In some embodiments, the reset command can reset the latest changes. In some embodiments, the reset command can reset several changes. In some embodiments, the reset command can reset the stimulation parameters back to the parameters specified in the currently executing stimulation program.

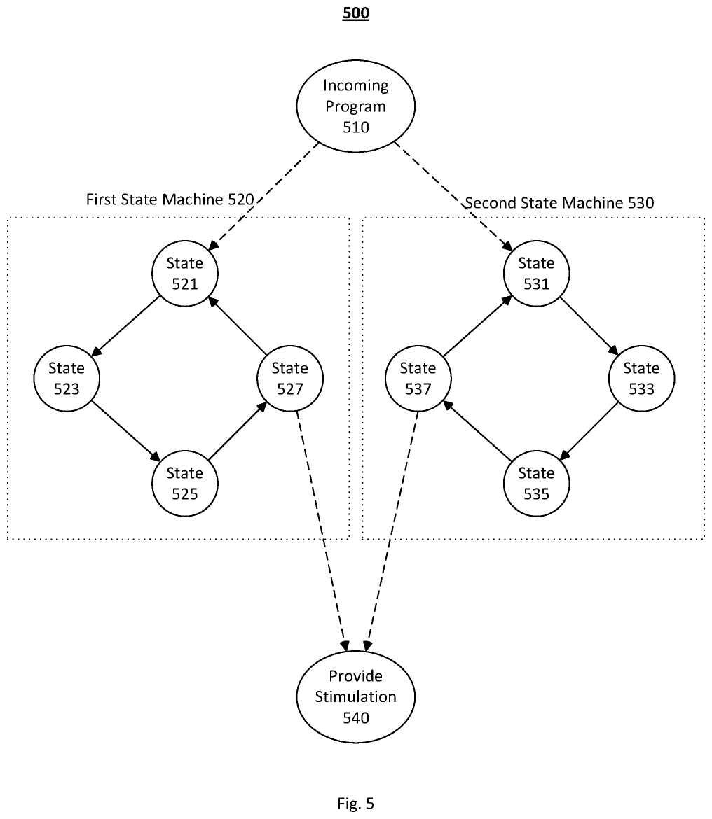

FIG. 5 depicts an exemplary logical diagram 500 of state machines, consistent with disclosed embodiments. Diagram 500 shows the relationship between the state machines and an incoming stimulation program, and the relationship between the state machines and the stimulation provided. A pulse generator (e.g., IPG 110, pulse generator 200, or the like) can implement state machines consistent with diagram 500 to enable the pulse generator to receive and store a second stimulation program while providing output according to a first stimulation program. As may be appreciated, the stimulation provided can be used for neuromodulation.

Diagram 500 depicts an exemplary implementation including two state machines, first state machine 520 and second state machine 530. In some embodiments, each of these state machines can be associated with a program memory (e.g., program memory 220, 230). In some embodiments, each state machine can be configured with four states. As may be appreciated, the disclosed embodiments are not limited to state machines including four states. In some embodiments additional states may be included, or fewer states.

In some embodiments, when a state machine (e.g., state machine 520 or state machine 530) is in the first state (e.g., state 521 or state 531), the corresponding program memory can be accessible for writing. A pulse generator can obtain and load the stimulation program into the program memory in response to an update request. As described herein, the update request can specify or include the stimulation program. In response to the update request, the pulse generator can cause the state machine to transition to the second state (e.g., state 523, or state 533).

In some embodiments, the pulse generator can write the stimulation program to the corresponding program memory when the state machine is in the second state. Once the pulse generator has completed writing the stimulation program to the corresponding program memory, the pulse generator can cause the state machine to transition to the third state (e.g., state 525, or state 535).

In some embodiments, when the state machine is in the third state, the corresponding program memory can be accessible for reading. In response to an execution request, the pulse generator can cause the state machine to transition to the fourth state. The state machine can be in the fourth state (e.g., state 527, or state 537) when the stimulation program is read from the corresponding program memory. The pulse generator can be configured to provide stimulation 540 according to the stimulation program read from the corresponding program memory.

Consistent with disclosed embodiments, an incoming program 510 can be obtained from a device (e.g., controller 130 or programmer 140). In some embodiments, the incoming program can be stored in a program memory corresponding to a state machine in the first state (e.g., state 521 or state 531). In some embodiments, both state machines may be in the first state. In such embodiments, the pulse generator can store the incoming program in either program memory and update the corresponding state machine to the second state. In various embodiments, as described herein, state machines can possess additional transitions. These additional transitions can ensure that only one state machine can be in state one. For example, when a first state machine is in state one and the second state machine is in state four, the second state machine can transition back to state three, rather than transitioning to state one. In some embodiments, the addition transitions can ensure that only one state machine can be in state three. For example, when a first state machine is in state three and the second state machine is in state four, the second state machine can transition back to state one, rather than transitioning to state three. As an additional example, when a first state machine is in state three and the second state machine is in state two, the first state machine can transition to state one in response to the second state machine transitioning to state three.

In some embodiments, the pulse generator can maintain an incoming program queue. The incoming program queue can be configured to temporarily store stimulation programs until a program memory becomes available (e.g., the associated state machine transitions to state one). The contents of the incoming program queue can then be written to the program memory.

The architecture described herein can support rapid changes in stimulation parameters without requiring cessation of stimulation. Consistent with the disclosed embodiment, a latency between receiving an update request and providing stimulation in response to the request can be less than 100 milliseconds. In some embodiments, such a latent can be less than 50 milliseconds.

In some embodiments, the pulse generator can be configured to execute stimulation programs without requiring an express execution request. In such embodiments, a state machine in state three can automatically transition to state four, provided another state machine is not already in state four. For example, when first state machine 520 is in state 527, the pulse generator can provide stimulation according to a first stimulation program stored in a program memory corresponding to first state machine 520. Second state machine 530 can complete responding to an update request and transition to state 535. Upon completion of stimulation according to the first stimulation program, the first state machine 520 can transition to state 521, while second state machine 530 can transition to state 537. The pulse generator can then provide stimulation according to a second stimulation program stored in a program memory corresponding to second state machine 530.

In some embodiments, the pulse generator can be configured to repeat execution of stimulation programs. In such embodiments, a state machine in state four can automatically return to state four, provided another state machine is not in state three. In such embodiments, the pulse generator can be configured to repeat execution of the stimulation program until an execution stop request is received.

The foregoing description has been presented for purposes of illustration. It is not exhaustive and is not limited to precise forms or embodiments disclosed. Modifications and adaptations of the embodiments will be apparent from consideration of the specification and practice of the disclosed embodiments. For example, the described implementations include hardware, but systems and methods consistent with the present disclosure can be implemented with hardware and software. In addition, while certain components have been described as being coupled to one another, such components may be integrated with one another or distributed in any suitable fashion.

Moreover, while illustrative embodiments have been described herein, the scope includes any and all embodiments having equivalent elements, modifications, omissions, combinations (e.g., of aspects across various embodiments), adaptations or alterations based on the present disclosure. The elements in the claims are to be interpreted broadly based on the language employed in the claims and not limited to examples described in the present specification or during the prosecution of the application, which examples are to be construed as nonexclusive. Further, the steps of the disclosed methods can be modified in any manner, including reordering steps or inserting or deleting steps.

The features and advantages of the disclosure are apparent from the detailed specification, and thus, it is intended that the appended claims cover all systems and methods falling within the true spirit and scope of the disclosure. As used herein, the indefinite articles “a” and “an” mean “one or more.” Similarly, the use of a plural term does not necessarily denote a plurality unless it is unambiguous in the given context. Further, since numerous modifications and variations will readily occur from studying the present disclosure, it is not desired to limit the disclosure to the exact construction and operation illustrated and described, and accordingly, all suitable modifications and equivalents may be resorted to, falling within the scope of the disclosure.

As used herein, unless specifically stated otherwise, the term “or” encompasses all possible combinations, except where infeasible. For example, if it is stated that a component may include A or B, then, unless specifically stated otherwise or infeasible, the component may include A, or B, or A and B. As a second example, if it is stated that a component may include A, B, or C, then, unless specifically stated otherwise or infeasible, the component may include A, or B, or C, or A and B, or A and C, or B and C, or A and B and C.

Other embodiments will be apparent from consideration of the specification and practice of the embodiments disclosed herein. It is intended that the specification and examples be considered as example only, with a true scope and spirit of the disclosed embodiments being indicated by the following claims.

What is claimed is:

1. A neuromodulation system, comprising:
an implantable pulse generator configured to provide neuromodulation to a patient based on a sequence of neuromodulation programs, the implantable pulse generator comprising alternately accessible memories; and
wherein
a second memory of the memories is configured for storing a second neuromodulation program of the sequence of neuromodulation programs while the implantable pulse generator provides neuromodulation according to a first neuromodulation program of the sequence of neuromodulation programs, the first neuromodulation program stored in a first memory of the memories.

2. The neuromodulation system of claim 1, wherein the sequence of neuromodulation programs is configured to enable one or more functions selected from a group consisting of postural and/or locomotor activity, upper and/or lower limb activity, voluntary voiding of the bladder and/or bowel, sexual function, autonomic control of cardiovascular function, respiratory and/or coughing function, body temperature control, and normalized metabolic processes.

3. The neuromodulation system of claim 1, wherein:
the implantable pulse generator is further configured to receive the first neuromodulation program and the second neuromodulation program from a programmer that stores the sequence of neuromodulation programs.

4. The neuromodulation system of claim 3, further comprising:
a controller configured to maintain a communication link with the implantable pulse generator.

5. The neuromodulation system of claim 4, wherein:
the implantable pulse generator is further configured to cease providing the neuromodulation when the communication link fails for a period of time.

6. The neuromodulation system of claim 5, wherein the period of time is in a range of 0.1 second to 5 minutes.

7. The neuromodulation system of claim 5, wherein the period of time is in a range of 0.1 second to 60 seconds.

8. The neuromodulation system of claim 1, wherein:
the implantable pulse generator is further configured to:
receive, from the programmer, an update instruction specifying an update of a stimulation parameter;
store, in a parameter memory, the update of the stimulation parameter;
access, in the parameter memory during provision of the neuromodulation according to a first neuromodulation program, the update of the stimulation parameter; and
provide a next pulse of the neuromodulation based on the stimulation parameter.

9. The neuromodulation system of claim 8, wherein the stimulation parameter comprises at least one of intensity, amplitude, pulse width, pulse ratio, electrode configuration, frequency, carrier frequency, or burst frequency.

10. The neuromodulation system of claim 1, wherein the second memory of the memories is configured for storing the second neuromodulation program according to a second state machine associated with the second memory, wherein:
when the second state machine is in a first state, the second memory is accessible for writing the second neuromodulation program;
the second state machine is in a second state when the second neuromodulation program is being written to the second memory;
when the second state machine is in a third state, the second memory is accessible for reading the second neuromodulation program; and
the second state machine is in a fourth state when the second neuromodulation program is being read from the second memory.

11. The neuromodulation system of claim 10, wherein:
the second state machine transitions from the first state to the second state in response to an update request;
the second state machine transitions from the second state to the third state upon completion of the update request; and
the second state machine transitions from the third state to the fourth state in response to an execution request upon completion of the neuromodulation provided according to the first neuromodulation program.

12. The neuromodulation system of claim 11, wherein a latency between receiving an update request and providing stimulation according to the request is less than 100 millisecond.

13. The neuromodulation system of claim 11, wherein a latency between receiving an update request and providing stimulation according to the request is less than 50 milliseconds.

14. The neuromodulation system of claim 10, wherein:
completion of the neuromodulation according to the first neuromodulation program depends upon a repeat value associated with the first neuromodulation program.

15. A method for providing neuromodulation according to a sequence of neuromodulation programs, comprising:
receiving, by the implantable pulse generator and during provision of neuromodulation according to a first neuromodulation program in the sequence of neuromodulation programs, the first neuromodulation program read from a first memory of the implantable pulse generator, a second neuromodulation program in the sequence of neuromodulation programs;
storing, in a second memory of the implantable pulse generator, the second neuromodulation program; and
providing, by the implantable pulse generator and upon completion of the provision of the neuromodulation according to the first neuromodulation program, neuromodulation according to the second neuromodulation program stored in the second memory.

16. The method of claim 15, wherein the first neuromodulation program and the second neuromodulation program are received from a programmer that stores the sequence of neuromodulation programs.

17. The method of claim 15, wherein the method further comprises:
receiving, by the programmer from a user, an instruction corresponding to the sequence of neuromodulation programs;
providing, by the programmer in response to the receipt of the instruction, the first neuromodulation program, and
wherein the instruction enables one or more functions selected from a group consisting of postural and/or locomotor activity, upper and/or lower limb activity, voluntary voiding of the bladder and/or bowel, sexual function, autonomic control of cardiovascular function, respiratory and/or coughing function, body temperature control, and normalized metabolic processes.

18. The method of claim 15, wherein the method further comprises:
a controller configured to maintain a communication link with the implantable pulse generator.

19. The method of claim 15, the method further comprises:
ceasing provision of the neuromodulation, by the implantable pulse generator, when the communication link fails for a period of time.

20. The method of claim 15, the method further comprises:
receiving, by the implantable pulse generator and during the provision of the neuromodulation according to the first neuromodulation program, an update instruction specifying an update of a stimulation parameter;
storing, in a parameter memory, the update of the stimulation parameter;
accessing, in the parameter memory during provision of the neuromodulation according to a first neuromodulation program, the update of the stimulation parameter; and
providing, by the implantable pulse generator, a next pulse of the neuromodulation based on the stimulation parameter.

21. The method of claim 20, wherein the stimulation parameter comprises at least one of intensity, amplitude, pulse width, pulse ratio, electrode configuration, frequency, carrier frequency, or burst frequency.

22. The method of claim 15, wherein:
storing the second neuromodulation program comprises storing the second neuromodulation program according to a second state machine associated with the second memory, the second state machine configured to:
enable writing to the second memory when the second state machine is in a first state; and
transition from the first state to a second state in response to an update request to write the second neuromodulation program to the second memory; and
providing the neuromodulation according to the second neuromodulation program comprises accessing the second memory according to the second state machine, the second state machine configured to:
enable access to the second memory when the second state machine is in a third state; and
transition from the third state to a fourth state in response to an execution request to provide neuromodulation according to the second neuromodulation program.

23. The method of claim 21, wherein the execution request is provided based on a repeat value associated with the first neuromodulation program.
